(19) United States
(12) Patent Application Publication
(10) Pub. No.: US 2008/0262330 A1
(43) Pub. Date: Oct. 23, 2008

(54) Analyte Sensors and Compositions for Use Therein

(76) Inventors: Melissa M. Reynolds, Ann Arbor, MI (US); Scott Merz, Ann Arbor, MI (US); Cortney White, Belleville, MI (US)

Correspondence Address: GOODWIN PROCTER LLP;PATENT ADMINISTRATOR, EXCHANGE PLACE, BOSTON, MA, 02109-2881, US

(21) Appl. No.: 11/994,003

(22) PCT Filed: Jun. 30, 2006
(86) PCT No.: PCT/US06/25856

§ 371 Date: Jul. 2, 2008

Related U.S. Application Data
(60) Provisional application No. 60/695,265, filed on Jun. 30, 2005.

(51) Int. Cl. A61B 5/1486

(52) U.S. Cl. 600/347

(57) ABSTRACT

The present invention relates to analyte sensors and compositions, membranes for use in analyte sensors. For example, an analyte sensor is provided that comprise an electrode surface comprising an enzyme; an a biocompatible analyte permeable composition comprising a nitric oxide generating agent.

### RELATED APPLICATIONS This application claims priority to U.S. Ser. No. 60/695,265 filed Jun. 30, 2005 and hereby incorporated by reference in its entirety.

### INTRODUCTION

Enzymatic biosensors have been used for over 25 years to clinically monitor analyte levels, such as glucose. clinically. In general, the sensors are composed of an enzyme layer, a permselective layer, used to eliminate interferences such as ascorbate, and an outer polymeric layer that provides a biocompatible interface and controls analyte mass transfer to the enzyme layer beneath.

The outer interface is typically prone to biofouling. For example, adhesion of proteins and platelets to the sensor surface may cause inaccurate measurements of the true levels of species in the bulk blood or in bulk tissue. Due at least in part to the metabolic activity of the biolayer, or accumulation of proteins, cell and other biological materials, analyte readings such as oxygen and glucose readings are low, and carbon dioxide readings are high compared to that of, for example, the bulk blood measured by standard blood draw measurements. This biofouling may be one of the major contributions leading to decreased reliability of such sensors. For example, leukocytes can migrate out of the circulatory system and adhere to an implanted device. This leukocyte accumulation may damage the sensor and/or compromise the accuracy of the glucose readings.

Typically, blood or tissue analyte levels, such as for example, glucose levels, are performed clinically using, for example, discrete blood draws which are then analyzed for glucose in a remote laboratory using a bench top sensor. Not only are multiple blood draws required to monitor a patient's glucose levels as a function of time, but time delays between blood draws and the measurement do not immediately provide the physician with the result of the actual glucose measurement. This delay may be crucial for treating a patient in an appropriate timeframe. Further, in the case of glucose monitoring of a diabetic patient, for example, an optimized insulin therapy is known to reduce the risk of chronic complications as well as optimizing metabolic control during surgical procedures, intensive care or dialysis treatment.

A need exists for a devices and methods that allow for continuous and/or in vivo and/subcutaneously measured analyte levels, such as glucose level measurements of a patient's blood. In the case of glucose, such devices and methods would provide health care professionals with the information required to effectively manage their patient's glucose levels thereby optimizing insulin therapy.

### SUMMARY OF THE INVENTION

It is an object of the invention to provide sensors and methods for detecting analytes of bodily fluids.

In an embodiment, a an analyte sensor is provided that comprises an electrode surface comprising an enzyme; and a biocompatible analyte permeable composition comprising a nitric oxide generating agent. In some embodiments, the enzyme is an oxidase, for example glucose oxidase. In other embodiments, the enzyme oxidizes glucose and generates hydrogen peroxide. Such sensors may be implantable in a patient and/or capable of continuously measuring blood glucose levels in a patient for 3 days or more. In some embodiments, a sensor disclosed herein is subcutaneously implantable.

The biocompatible analyte permable composition may be disposed on an electrode surface of a sensor. Such an analyte permeable composition may, in some embodiments, substantially exclude at least one of: ascorbate, urate, cysteine or paracetamol. In one embodiment, an analyte permeable composition is substantially glucose permeable.

Nitric oxide generating agent may be selected from one or more of a metal-ligand complex, where, in some embodiments, a nitric oxide generating agent is metal capable of reducing nitrosothiol to generate NO and the composition further includes a ligand.

A metal ligand complex may be a metal-cyclen complex or a metal-cyclam complex, for example, a a copper-cyclen complex or a copper-cyclam complex. In some embodiments, a metal-ligand complex is a N₄ donor type macrocycle.

The nitric oxide generating agent may be, for example, represented by:

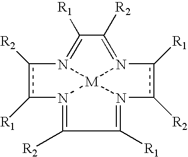

wherein:
R₁ and R₂ represent each independently: alkyl, alkenyl, alkynl, H, or any R₁ and R₂ together form an aryl or an heteroaryl;
------- represents a bond with any bond order;
M is a metal; and salts thereof.

In some embodiments, M is metallic ion selected from the group consisting of Ca, Cu, Zn, Co, Mn, Al, Fe, V, Cr), and Ti. A metallic ion may be redox active. For example, M may be Cu(II).

Salts of nitric oxide generating compounds are also contemplated by the invention, and may be for example, an pharmaceutically acceptable salt, and/or a Cl, I, F, or Br salt. In some embodiments, the nitric oxide generating agent is selected from the group consisting of Cu(II)-dibenzo[e,k]-2,3,8,9-tetraphenyl-1,4,7,10-tetraaza-cyclododeca-1,3,7,9-tetraene, Cu(II) dibenzo[e,k]-2,3,8,9-tetramethyl-1,4,7,10-tetraaza-cyclododeca-1,3,7,9-tetraene, or Cu(II) dibenzo[e,k]-2,3,8,9-tetraethyl-1,4,7,10-tetraaza-cyclododeca-1,3,7,9-tetraene.

A biocompatible membrane for use in an analyte sensor comprising a nitric oxide generating agent, wherein said analyte sensor is capable of continuously measuring analyte levels such as blood glucose levels for 3 days or more is also contemplated by this invention. For example, a biocompatible membrane may comprise a first layer comprising immobilized glucose oxidase; and a second layer disposed on the first layer, wherein said second layer comprises a metal-ligand complex.

Biocompatible membrane disclosed herein may include a membrane with a thickness of about 0.1 μm to about 15 μm.

A method for detecting glucose levels in the blood of a patient over 2 days or more is also disclosed, wherein said method comprises instilling a glucose sensor that comprises a biocompatible analyte permeable composition subcutaneously into a patient; wherein said composition comprises a nitric oxide generating agent.

The disclosure also provides for a glucose detection kit comprising a glucose sensor comprising a nitric oxide generating agent; and instructions for use. The use of a nitric oxide generating agent for a blood glucose sensor is also contemplated by this disclosure.

The present invention provides a number of methods of making the subject compositions. Examples of such methods include those described in the Exemplification below.

The embodiments and practices of the present invention, other embodiments, and their features and characteristics, will be apparent from the description, drawings and claims that follow.

### BRIEF DESCRIPTION OF THE FIGURES

FIG. 1 illustrates NO-generation at a Cu(II)-polymer/blood interface from endogenous S-nitrosothiols.

FIG. 2 depicts a schematic design of an embodiment of an amperiometric glucose sensor contemplated by this disclosure.

FIG. 3 depicts a two layer membrane for use with a glucose sensor.

FIG. 4 shows a NO microsensor configuration used to measure NO surface concentrations of (a) polymer films and the bulk blood (b) fresh porcine blood or PBS buffer, and (c) depicts polymers films with Cu(II)-ligand complex (c) or control films (d).

FIG. 5 depicts sham catheters implanted within arteries of swine for 8 h, with (a) and without (b) NO generating coatings. Region to left of dashed line was exposed to flowing blood.

FIG. 6 depicts the NO generating profile for Tecophilic-SP-80A-150 film containing 4% wt. of CuDTTCT using GSH and GSNO after 2 days.

FIG. 7 depicts the NO generating profile for Tecophilic-SP-80A-150 film containing 4% wt. of CuDTTCT using GSH and GSNO after 3 days.

FIG. 8 depicts the NO generating profile for Tecophilic-SP-80A-150 film containing 4% wt. of CuDTTCT using GSH and GSNO.

FIG. 9 depicts the NO generating profile for Tecophilic-SP-80A-150 film containing 8% wt. of CuDTTCT using GSH and GSNO.

FIG. 10 depicts the NO generating profile for tecophilic polyurethane containing 4% wt. of CuDTTCT after 1 month.

### DETAILED DESCRIPTION OF THE INVENTION

### Overview

The present invention relates at least in part, to analyte sensors and compositions for use in analyte sensors such as, for example, glucose sensors.

The compositions of the present invention may be adapted for application for use in an analyte sensor, such as a glucose sensor. In certain embodiments, the subject compositions of the present invention are understood to exert their effect in part by contact with a portion of a bodily fluid being tested, such as for example, blood. Contact refers to a physical touching, either directly with the subject composition being applied without intervening barrier to the bodily fluid being tested or indirectly, where the subject composition is applied to or is formed on a surface of an interposed material, passing therethrough to come into direct contact with the bodily fluid being tested. Contact, as used herein, includes those situations where the agents of the present invention are initially positioned to contact the bodily fluid being tested, and those situations where the agents of the present invention are initially positioned in proximity to the bodily fluid being tested without contacting it, and subsequently move, migrate, flow, spread or are transported to enter into contact with the bodily fluid being tested. The compositions of the invention may be formed as a solid object, or as part of a sensor that can be implantable in the anatomic area, or as a film or mesh that may be used to cover a segment of sensor. A variety of techniques for implanting solid objects and sensors in relevant anatomic areas will be likewise familiar to practitioners of ordinary skill in the art.

### Definitions

For convenience, before further description of the present invention, certain terms employed in the specification, examples, and appended claims are collected here. These definitions should be read in light of the remainder of the disclosure and understood as by a person of skill in the art. Also, the terms “including” (and variants thereof), “such as”, “e.g.” as used herein are non-limiting and are for illustrative purposes only.

The articles “a” and “an” are used herein to refer to one or to more than one (i.e. to at least one) of the grammatical object of the article. By way of example, “an element” means one element or more than one element.

The term “access device” is an art-recognized term and includes any medical device adapted for gaining or maintaining access to an anatomic area. Such devices are familiar to artisans in the medical and surgical fields. An access device may be a needle, a catheter, a cannula, a trocar, a tubing, a shunt, a drain, an endoscope, or any other device adapted for use in a vein, artery, cranial, spinal, and brain areas, or any other medical device suitable for entering or remaining positioned within the preselected anatomic area.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1M NaOH at 37° C. until complete degradation is observed. The solution is then neutralized with 1M HCl. About 200 μL of various concentrations of the degraded sample products are placed in 96-well tissue culture plates and seeded with human gastric carcinoma cells (GT3TKB) at 10⁴/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biofouling” refers to the attachment or association of biological cells, proteins, or other biological based compounds with or on a surface that is in contact with a bodily fluid for a period of time.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, nitric oxide generating agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The terms “incorporated” and “encapsulated” are art-recognized when used in reference to an nitric oxide generating agent (or other material) and a polymeric composition, such as a composition of the present invention. In certain embodiments, these terms include incorporating, formulating or otherwise including such agent into a composition which allows for the prevention of biofouling and/or permits analyte diffusion of such agent in the desired application. The terms may contemplate any manner by which an nitric oxide generating agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a nitric oxide generating agent or other material and at least one other agent or other material in a subject composition.

More specifically, the physical form in which any nitric oxide generating agent or other material is encapsulated in polymers may vary with the particular embodiment. For example, a nitric oxide generating agent or other material may be first encapsulated in a microsphere and then combined with the polymer in such a way that at least a portion of the microsphere structure is maintained. Alternatively, a nitric oxide generating agent or other material may be sufficiently immiscible in the polymer of the invention that it is dispersed as small droplets, rather than being dissolved, in the polymer. Any form of encapsulation or incorporation is contemplated by the present invention, in so much as the effectiveness over time of any encapsulated nitric oxide generating agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyl trihexyl citrate (c. 20 weight percent or less), butyl benzyl phthalate, dibutyl phthalate, dioctylphthalate, n-butyryl tri-n-hexyl citrate, diethylene glycol dibenzoate (c. 20 weight percent or less) and the like.

As used herein, the term “nitric oxide” encompasses uncharged nitric oxide and charged nitric oxide species, including for example, nitrosonium ion and nitroxyl ion.

The term “metal-ligand complex” refers to a chemical species with at least one ligand coordinated to at least one central metal ion.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g.; the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and include both unsubstituted and substituted amines, e.g., a moiety that may be represented by the general formulas:

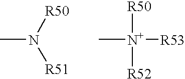

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

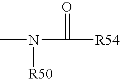

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

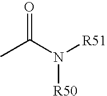

wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

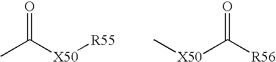

wherein X50 is a bond or represents an oxygen or a sulfur, and R55 represents a hydrogen, an alkyl, an alkenyl, —(CH₂)ₘ-R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The definition of each expression, e.g. alkyl, m, n, etc., when it occurs more than once in any structure, is intended to be independent of its definition elsewhere in the same structure unless otherwise indicated expressly or by the context.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2nd ed., Wiley, N.Y., (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described above include such materials which otherwise correspond thereto, and which have the same general properties thereof (e.g., biocompatible, nitric oxide generating), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

### EXEMPLARY SUBJECT MATERIALS

A variety of nitric oxide generating agents are contemplated by the present invention. Practitioners of the art will readily appreciate the circumstances under which various nitric oxide agents are appropriate for use in analyte sensors. For example, as described in the Exemplification section below, copper cyclen complexes can be used to substantially prevent biofouling on glucose sensors.

Nitric oxide generating agents are defined herein to include only those agents that do not have covalently attached nitric oxide releasing moieties, rather, nitric oxide generating agents are capable of generating nitric oxide when in contact with nitrosothiols, such as those found in bodily fluids such as blood.

For example, nitric oxide generating agents include metal-ligand complexes. For example, metal-ligand complexes include complexes that have a neutral carrier type ligand with a high metal binding affinity. In some embodiments, such ligands have a high binding affinity for copper. Metal-ligand complexes may, in some embodiments, have a planar square-type geometry which, in some embodiments, may provide a minimum amount of steric hindrance to the approach of an electron source to the center metal of the complex so that the metal ion can easily be reduced. Ligand complexes include nitrogen or sulfur donating compounds, such as Nₓ-donor macrocyclic ligands (x=2, 3, 4, 5, 6, 7, 8) such as cyclen, cyclam and their derivatives, and crown ethers and S_{y}-donor macrocyle-type ligands (y=2, 3, 4, 5, 6, 7, 8). In an embodiment, a metal-ligand macrocycle is a N₄ macrocycle.

Examples of a metal-cyclen complex includes those metal complexes that include the structure:

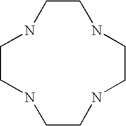

and derivatives of this cyclen ligand. Metal-cyclam structures include structures such as:

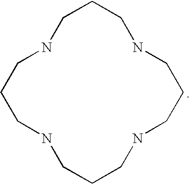

For example, metal-cyclam structures include 1,8 bis(pyridylmethyl)cyclam, 1,11-bis(pyridylmethyl)cyclam, and diooxocyclam ligands and structural isomers thereof. These include multi-amine substrates that can be aromatic or aliphatic.

Exemplary ligands include dibenzo[e,k]-2,3,8,9-tetraphenyl-1,4,7,10-tetraaza-cyclododeca-1,3,7,9-tetraene; dibenzo[e,k]-2,3,8,9-tetramethyl-1,4,7,10-tetraaza-cyclododeca-1,3,7,9-tetraene; dibenzo[e,k]-2,3,8,9-tetraethyl-1,4,7,10-tetraaza-cyclododeca-1,3,7,9-tetraene, and/or salts thereof. Such ligands can be modified to include halogen atoms.

Other non-limiting examples of nitric oxide generating agents include, in general, enzymes having nitrate, nitrite, nitrosothiol reductase activity, for example, xanthine oxidase and nitrite and/or nitrate reductases derived from plants or bacteria. Nitric oxide generating agents may include hydrogel metal complexes. In an alternate embodiment, the nitric oxide generating agent may be metals and/or metal ions, for example, calcium, magnesium, cobalt, copper, manganese, iron, molybdenum, tungsten, vanadium, aluminum, chromium, zinc, nickel, platinum, tin, ions thereof, and/or mixtures thereof. Nitric oxide generating agents may include copper (II) phosphate and various copper salts. In some embodiments, the metal entity in a metal-ligand complex may be associated with a ligand either, for example within the ligand or outside the ligand. The metal entity in or associated with a ligand includes such metals as calcium, magnesium, cobalt, copper, manganese, iron, molybdenum, tungsten, vanadium, aluminum, chromium, zinc, nickel, platinum, tin, ions thereof, and/or mixtures thereof.

Without being limited to any theory, nitric oxide generating agents, when exposed to endogenous or exogenous sources of nitrates, nitrites, or nitrosothiols, and optionally in the presence of reducing agents, generates an active metal (for example, with coordination(I)) species that generates NO within or at the surface of a composition. It is to be understood that the sources of nitrates, nitrites, nitrosothiols and reducing agents may be from bodily fluids such as blood, within the composition, within the sensor, and/or may be injected intravenously or otherwise added or administered to the bodily fluid of interest.

The nitric oxide generating agents contemplated herein may decompose at a temperature that is higher than a typical processing temperature for the manufacture of analyte sensors, and/or at a higher temperature than a nitric oxide releasing agent. For example, the nitric oxide generating agents may decompose at a temperature above about 100° C., or even above about 125° C. In an embodiment, the nitric oxide generating agents contemplated by this disclosure are thermally stable.

Nitric oxide releasing agents are defined herein to include agents that have nitric oxide donor moieties covalently attached or otherwise bonded to the agent. Non-limiting examples of nitric oxide releasing agents include such agents as S-nitrosothiols, S-nitroso amino acids, S-nitroso-polypeptides, and nitrosoamines. One group of such nitric oxide donor moieties include the S-nitrosothiols, which are compounds that include at least one —S—NO group. Such compounds include S-nitroso-polypeptides (the term “polypeptide” includes proteins and also polyamino acids that do not possess an ascertained biological function, and derivatives thereof); S-nitrosylated amino acids(including natural and synthetic amino acids and their stereoisomers and racemic mixtures and derivatives thereof); S-nitrosated sugars, S-nitrosated-modified and unmodified oligonucleotides; and an S-nitrosated hydrocarbon where the hydrocarbon can be a branched or unbranched, and saturated or unsaturated aliphatic hydrocarbon, or an aromatic hydrocarbon; S-nitroso hydrocarbons having one or more substituent groups in addition to the S-nitroso group; and heterocyclic compounds. S-nitrosylated proteins include thiol-containing proteins(where the NO group is attached to one or more sulfur group on an amino acid or amino acid derivative thereof) from various functional classes including enzymes, such as tissue-type plasminogen activator(TPA) and cathepsin B; transport proteins, such as lipoproteins, heme proteins such as hemoglobin and serum albumin; and biologically protective proteins, such as the immunoglobulins and the cytokines. Other suitable S-nitrosothiols that are S-nitroso-angiotensin converting enzyme inhibitors.

Nitric oxide donor agents include compounds that include at least one —O—NO group. Such compounds include O-nitroso-polypeptides(the term “polypeptide” includes proteins and also polyamino acids that do not possess an ascertained biological function, and derivatives thereof); O-nitrosylated amino acids (including natural and synthetic amino acids and their stereoisomers and racemic mixtures and derivatives thereof); O-nitrosated sugars; O-nitrosated-modified and unmodified oligonucleotides; and an O-nitrosated hydrocarbon where the hydrocarbon can be a branched or unbranched, saturated or unsaturated aliphatic hydrocarbon, or an aromatic hydrocarbon; O-nitroso hydrocarbons having one or more substituent groups in addition to the O-nitroso group; and heterocyclic compounds.

Further nitric oxide donor agents include nitrites which have an —O—NO group wherein R is a protein, polypeptide, amino acid, branched or unbranched and saturated or unsaturated alkyl, aryl or a heterocyclic. N-nitrosoamines, which are compounds that include at least one —N—NO group, C-nitroso compounds that include at least one —C—NO, and compounds that include at least one —O-NO₂ group.

Also contemplated by this disclosure as nitric oxide donor agents are diazeniumdiolates, such as those represented by:

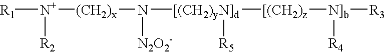

where d and b are independently selected from 0 or 1; R₁, R₂, R₃, R_{4,} R₅ are independently selected from hydrogen, C₃₋₈ cycloalkyl, C₁₋₁₂ straight or branched chain alkyl, benzyl, benzoyl, phthaloyl, acetyl, trifluoroacetal, p-tolyl, t-butooxycarbonyl, or 2,2,2-trichloro-t-butoxycarbonyl; z, x, and z are independently selected from an integer between 2 and 13 inclusive; and salts thereof.

Such diazeniumdiolates include lipophilic dialkyldiamine diazeniumdiolates such as compounds with the structure RN[N(O)NO]⁻(CH₂)₆NH₂⁺R, where R may be, for example, CH₃, CH₂CH₃, (CH₂)₂CH₃, (CH₂)₃CH₃, (CH₂)₄(CH₃), (CH₂)₅CH₃, and (CH₂)₁₁CH₃.

### Polymers

A variety of polymers may be used in the subject invention. A polymer for such use may be biocompatible. As discussed below, the choice of polymer will depend in part on a variety of physical and chemical characteristics of such polymer and the use to which such polymer may be put.

Representative natural polymers include proteins, such as zein, modified zein, casein, gelatin, gluten, serum albumin, or collagen, and polysaccharides, such as cellulose, dextrans, hyaluronic acid, and polymers of alginic acid.

Representative synthetic polymers include polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyphosphates, polyesters, and polyurethanes. For example, polymers may include polydimethylsiloxane, ethylene vinyl acetate, nylons, polyacrylics, polymethyl methacrylate, polyethylenes, polypropylenes, polystyrenes, poly(vinyl chloride) (PVC), and polytetrafluoroethylene (PTFE). Silicon rubbers may also be used as a polymer.

Synthetically modified natural polymers include alkyl celluloses, hydroxyalkyl celluloses, cellulose ethers, cellulose esters, and nitrocelluloses. Other like polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

In some embodiments, compositions of this disclosure include a biocompatible polymer. Examples of biocompatible polymers include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonates), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Polyurethanes, silicones, and polyesters may be used as well aspolyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Polymers that resist protein adsorption may also be used in compositions contemplated by this disclosure. Such polymers include polyethylene glycols, polyurethanes and silicone elastomer, silica containing polymers, and poly(vinyl)chlorides.

Other polymers that may be used include tecophilic polyurethanes, PDMS co-polymers, carbamates, and the like. In some embodiments, polymers that regulate water up take may be used in the disclosed composition. Polymers contemplated by this disclosure may include those polymers that control the diffusion of nitric oxide generating agent, and/or polymers that control the diffusion of S-nitrosothiols.

All of the subject polymers may be provided as copolymers or terpolymers. These polymers may be obtained from chemical suppliers or else synthesized from monomers obtained from these suppliers using standard techniques.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have average molecular weights ranging from about 2000 or less to about 10,000,000 or more. Number-average molecular weight (Mn) may also vary widely, but generally fall in the range of about 1,000 to about 10,000,000. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

A flexible polymer may be used in the fabrication of a solid article. Flexibility involves having the capacity to be repeatedly bent and restored to its original shape. Solid articles made from flexible polymers are adapted for placement in anatomic areas where they will encounter the motion of adjacent organs or body walls. Certain areas of motion are familiar to practitioners dealing with implantable sensors. A flexible solid article can thus be sufficiently deformed by those moving tissues that it does not cause tissue damage. Flexibility is particularly advantageous where a solid article might be dislodged from its original position and thereby encounter an unanticipated moving structure; flexibility may allow the solid article to bend out of the way of the moving structure instead of injuring it. Such a flexible article might be suitable for inserting into pulsatile vessels such as the internal carotid artery, the cerebral arteries, the middle meningeal artery, the basilar artery, the vertebral artery, and the spinal arteries, or for inserting into more delicate structures in the head such as the venous sinuses that may also be affected by local movements. Use of a solid article according to the present invention in the aforesaid ways may allow less extensive dissections to be carried out with surgical preservation and protection of structures important to function. Solid articles may be configured as three-dimensional structures suitable for implantation in specific anatomic areas. For example, a solid article, such as a sensor, or implanted subcutaneously, or may be implantable into the margins of a resected bone or cartilaginous structure and may be fabricated according to the present invention to carry nitric oxide generating agent. Solid articles such as a sensor or membrande may be formed as films, meshes, sheets, tubes, or any other shape appropriate to the dimensions and functional requirements of the particular anatomic area. Physical properties of polymers may be adjusted to attain a desirable degree of flexibility by modification of the chemical components and crosslinking thereof, using methods familiar to practitioners of ordinary skill in the art.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, and ethyl acetate.

The mechanical properties of the polymer may be important for the processability of making molded or pressed articles for implantation or for use as a coating or layer. For example, the glass transition temperature may vary widely but must be sufficiently lower than the temperature of decomposition to accommodate conventional fabrication techniques, such as compression molding, extrusion or injection molding.

### Sensor Compositions and Membranes

Compositions suitable for use in an analyte sensor, in for example, a sensor membrane, include compositions comprising a biocompatible analyte permeable composition that includes a nitric oxide generating agent. Such biocompatible analyte permeable compositions may be suitable for use as a layer or membrane, disposed, at least in part, on a sensing layer or on an electrode surface of an analyte sensor. In part, a biocompatible analyte permeable composition of the present invention useful for use in analyte detection includes: (a) nitric oxide generating agent, and (b) a biocompatible polymer that is at least partially permeable to the analyte(s) of interest.

In certain embodiments, a nitric oxide generating agent is incorporated into a polymer, resulting in a composition or membrane suitable as a biocompatible analyte permeable composition. The nitric oxide generating agent may be covalently attached to the polymer, dispersed throughout the polymer, or disposed on the surface of a polymeric layer or membrane.

For a biocompatible analyte permeable composition, the nitric oxide generating agent or substance may added to a polymer or a composition comprising a polymer. A variety of methods are known in the art for encapsulating a substance in a polymer. For example, the agent or substance may be dissolved to form a homogeneous solution of reasonably constant concentration in the polymer composition, or it may be dispersed to form a suspension or dispersion within the polymer composition at a desired level of “loading” (grams of biologically active substance per grams of total composition including the agent, usually expressed as a percentage). For example, the nitric oxide generating agent may comprise 0.01%, 1%, 3% or even 5% or more by weight of a composition.

Suitable compositions may also comprise a wide range of additional materials. Fore example, materials may be incorporated into the compositions that alter the physical and chemical properties, including for example, the capability of preventing biofouling of the resulting composition and/or the analyte permeability of the composition. The composition may include materials or components that protect the metal sites on the composition. Without being limited thereto, such materials may include diluents, binders and adhesives, lubricants, disintegrants, colorants, bulking agents, flavorings, sweeteners, and miscellaneous materials such as buffers and adsorbents, in order to prepare a particular medicated composition, with the condition that none of these additional materials will interfere with the intended purpose of the subject composition.

In addition to the nitric oxide generating agent, the subject compositions may contain therapeutic agents. Any therapeutic agents in a subject composition may vary widely with the purpose for the composition. The term therapeutic agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment. Compositions contemplated by this disclosure can include one or more nitric oxide releasing agents alone or in combination with one or more nitric oxide generating agents.

Other non-steroidal anti-inflammatory drugs (i.e., aspirin, ibuprofen, naproxyn, ketoprofen and the like) or anti-inflammatory lymphokines (e.g., cyclosporine) may also be advantageously incorporated into a biocompatible analyte permeable composition. In addition, drugs that impede cell replication (e.g., antineoplastic agents) may be incorporated, such as vinca alkaloids (vincristine and vinblastine), taxol and taxol derivatives and other well-known anti-tumor drugs.

In an embodiment, the composition may include lipophilic salts of nitrite/nitrate or nitrosothiols within its matrix to create a reservoir of nitrite/nitrate or nitrosothiol that, for example, can continuously leak to a surface.

Compositions of this disclosure may also include other agents that assist prevention of biofouling or microbial interference. Such agents include antifungals and antibiotics. For example, gentamycin and/or penicillin, and/or other broad-spectrum antibiotics and antifungals (e.g., ketaconazole) can be incorporated into the enzyme mixture to prevent microbial growth.

Plasticizers and stabilizing agents known in the art may be incorporated in polymers of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results. For example, fillers, such as bovine serum albumin (BSA), mouse serum albumin (MSA), or silica particles, may be associated with or within the polymer matrix. In certain embodiments, the amount of filler may range from about 0.1 to about 50% or more by weight of the polymer matrix, or about 2.5, 5, 10, 25, 40 percent. Other fillers known to those of skill in the art, such as carbohydrates, sugars, starches, saccharides, celluoses and polysaccharides, including mannitose and sucrose, may be used in certain embodiments in the present invention. Buffers, acids and bases may be incorporated in the subject compositions to adjust their pH.

The charge, lipophilicity or hydrophilicity of any subject polymeric matrix may be modified by attaching or incorporating in some fashion an appropriate compound to the surface of a composition or membrane. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders. Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of a membrane or composition. Three exemplary types of coatings are seal, gloss and enteric coatings. Other types of coatings having various dissolution or erosion properties may be used to further modify subject matrices behavior, and such coatings are readily known to one of ordinary skill in the art.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, silica particles, and olefinic microfibrils. The amount of each of these optional additives employed in the composition is an amount necessary to achieve the desired effect.

Both nitric oxide generating agents and/or nitric oxide releasing agents can be used as part of a biocompatible analyte permeable composition. The use of solely nitric oxide releasing agents within the composition may result in a finite reservoir of donor within a given composition for use as a sensor membrane. To achieve higher or longer levels of NO release using nitric oxide releasing agents thicker coatings, for example, about 120-200 μm coatings may be required to achieve longer release. Sensors such as glucose sensors, however, may require a thin membrane in order to have reasonable diffusion rates of analyte through the membrane. In some embodiments, a membrane that includes compositions of this disclosure include membranes with a thickness of about 1 μm to about 100 μm, 1 μm to 50 μm, 1 μm to about 20 μm, or even 10 μm to about 50 μm. In other embodiments, a biocompatible analyte permeable composition that includes nitric oxide generating agents may release NO over a time greater than 1 day release or greater than 2, or even 3 day release or more. In some embodiments, the release of NO over 1, 2, or even 3 days or more is at a flux of 10×10⁻¹⁰ mol·cm⁻²·min⁻¹ under physiological conditions.

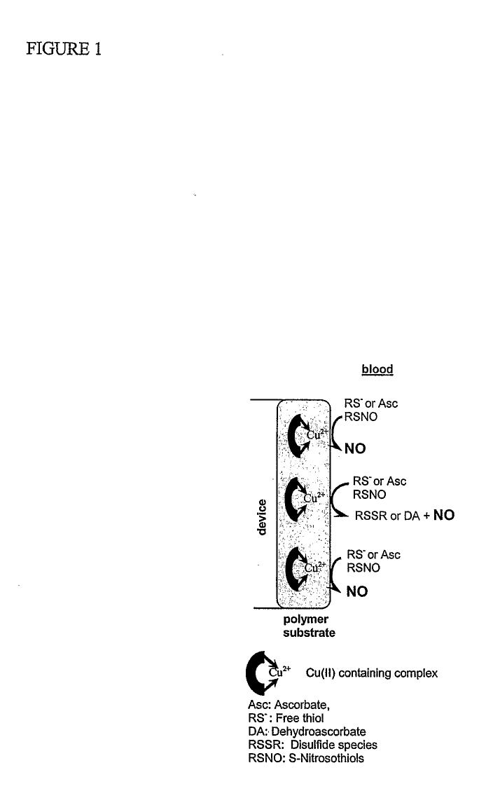

When a composition or membrane that includes a nitric oxide generating agent is placed in contact with blood, for example, it may facilitate the conversion of endogenous S-nitrosothiols to NO as shown schematically in FIG. 1. During normal hemostasis, S-nitrosothiols in the blood may interact with a composition disclosed herein to produce NO at the surface of the polymer or polymer coating. In this manner, generation of NO locally from the surface of the polymer may prevent platelet adhesion. The concentration of endogenous S-nitrosothiols found in human blood include S-nitrosoalbumin, 0.25-7 μm; S-nitrosoglutathione, 0.02-0.2 μm; S-nitrosocysteine, 0.2-0.3 μm; S-nitrosohemaglobin, 0.3(a) -0.003(v). A composition that includes a nitric oxide generating agent may be more biocompatible as compared to a composition that does not include such an agent.

Methods of making compositions disclosed herein include include dip coating a polymer or polymer composition in a solution or composition that includes nitric oxide generating or reducing agents, or by spray coating a polymer, polymer composition, or membrane with nitric oxide generating or reducing agents or a composition that includes such agents.

### Analyte Sensors

Generally, the present invention relates to analyte sensors having electrodes and a membrane that reduces analyte flux and substantially prevents biofouling. One contemplated embodiment is a sensor that includes a sensing layer disposed on a substrate and a biocompatible analyte permeable composition disposed over the sensing layer. Such a a biocompatible composition may include a nitric oxide generating agent and, in some embodiments, have enhanced biofouling characteristics as compared to a composition without a nitric oxide generating agent. In some embodiments, the disclosed sensors that include contemplated compositions will have fewer bio-fouling products adhered to the surface as compared to a sensor that does not contain nitric oxide generating compounds after 1, 2, 3 or more days of operation implanted a patient in-vivo or subcutaneously.

The disclosed sensors may be implantable for in-vivo use or subcutaneous use, or may be used externally on bodily fluids accessible without surgical or other invasive procedures. Alternatively, the disclosed sensors may be used on fluids analyzed remotely.

The present invention can be used in combination with other treatment modalities in certain embodiments. As examples, the sensors and methods of the present invention may be used in conjunction with surgery, with other sensors, or the sensor of the present invention may be capable of sensing more that one analyte simultaneously or in a step wise fashion, or may be used with systemic therapy, for example, insulin administration, or a combination of these modalities. For example, analyte sensors may be used in combination with a variable rate or programmable implantable insulin infusion pump.

Contemplated by this disclosure are analyte sensors such as those in contact with bodily fluids of a patient, such as those in contact with an interstitial space in a patient, or with blood contacted subcutaneously or in a vein or artery, saliva, urine, perspiration, and the like.

In an embodiment, a disclosed composition may be used with a analyte sensor such as a glucose sensor as a membrane that comprises, consists or consists essentially of two layers: a glucose oxidase layer, and layer that comprises a nitric oxide generating agent or a composition of this disclosure. In another embodiment, a membrane for use in a glucose sensor may include one or more layers, with at least one of the layers including a composition of this disclosure. The efficacy of sensing an analyte with a membrane, for example, a pharmaceutically acceptable membrane, that includes the subject composition may be greater as compared to the efficacy of a sensor without a nitric oxide generating agent or in a pharmaceutically acceptable membrane alone.

Electrodes for use in analyte sensors include those electrodes functioning on an amperometric basis. For example, a glucose sensor includes an electrode that comprises glucose oxidase that is substantially immobilized on the electrode surface. Such a sensor, when immersed in blood or interstitial fluid, provides a signal indicative of glucose concentration, and hence is useful as a blood glucose sensor in a variety of applications.

To provide an overall understanding, certain illustrative embodiments of an analyte sensor are described herein; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems or methods. Additionally, the shapes and sizes of components are also exemplary and unless otherwise specified, can be altered without affecting the scope of the disclosed and exemplary systems or methods of the present disclosure.

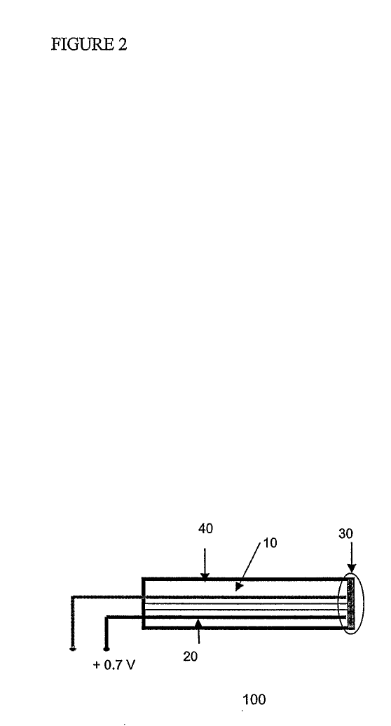

An exemplary embodiment of an analyte sensor is shown in FIG. 2. FIG. 2 depicts an amperiometric glucose sensor 100. The glucose sensor 100 includes an Ag cathode 10 and a Pt anode 20. The cathode 10 and anode 20 are substantially contained within a dual barrel glass capillary 40. The glucose sensor 100 includes a membrane of the instant invention 30.

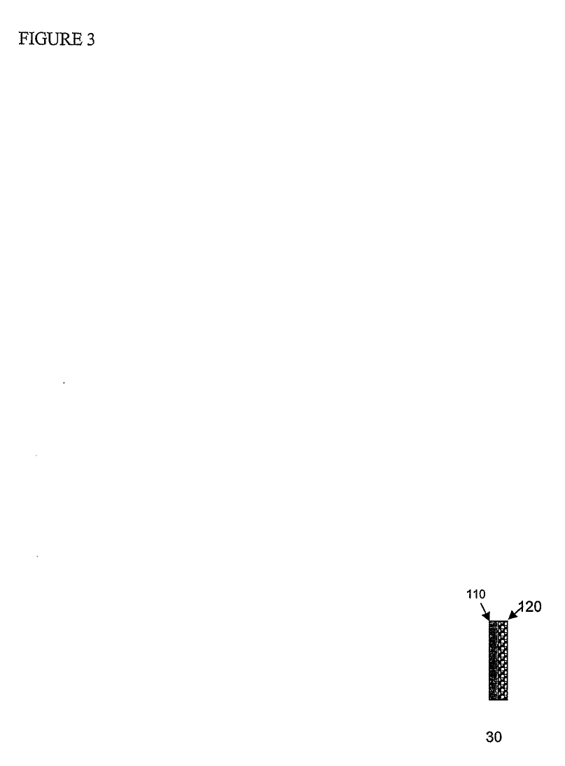

FIG. 3 depicts a two layer membrane 30 for use in an glucose sensor. Layer 110 comprises the immobilized glucose oxidase on the electrode surface depicted in FIG. 2. Layer 120 includes a composition of this invention that allows for glucose transport through the membrane, but substantially eliminates sensor interferences such as ascorbate, and also includes a biocompatible surface for use in a patient.

### EXEMPLIFICATION

The invention having been generally described, it will be more readily understood by reference to the following examples which are included merely for purposes of illustration of certain aspects and embodiments of the present invention and are not intended to limit the invention in any way.

### Example 1

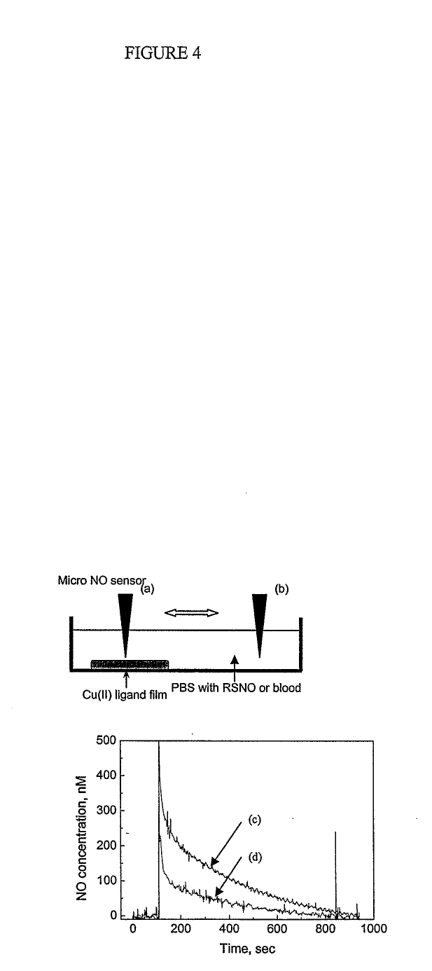

NO-generation from compositions using Cu(II) ligands is measured by injecting physiological levels of S-nitrosoglutathione and glutathione into a buffer. As FIG. 4 depicts, when only the S-nitrosothiol or the Cu(II)-containing polymer are present alone in the buffer, no detectable NO is generated. However, when the Cu(II)-containing polymer and the S-nitrosothiol are combined, NO is generated at surface concentrations found to be effective in preventing platelet adhesion in other studies.

To test the efficacy of NO-generation from the Cu(II) ligand CuDTTCT (Cu(II)-in blood, a NO electrochemical sensor (10 μm away from the polymer surface) is used to measure NO-generation at the surface of the material when immersed in blood. FIG. 84 shows that NO is generated at higher levels at the surface of the Cu(II) ligand material. When the sensor is removed from the surface of the Cu(II) ligand material and exposed to the bulk blood, minimal NO is detected, likely due to light decomposition of the S-nitrosothiols. The NO signal decreases with time as the S-nitrosothiol species is consumed; however, in flowing blood, there will be a fresh supply of the blood (and thus, S-nitrosothiols) at all times at the interface; thereby capable of generating NO for extended periods.

### Example 2

### Gross Thrombus Formation on Surface of Implanted Material

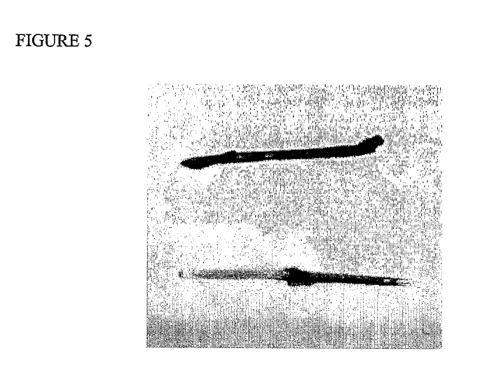

Cu(II) ligand containing polymers for coated sham sensors and control sensors are implanted in the femoral and jugular arteries of a porcine for 8 h and then explanted. Gross macroscopic images (see FIG. 5 for representative images) as well as SEM images show a platelet-free surface for the Cu(II) ligand polymer-coated sham sensors and a mature thrombus formation for the control sham sensors.

### Example 3

### Ambient and Thermal Stability

Using thermal gravimetric analysis, after heating the Cu(II)-materials past temperatures required for extrusion of common polymers, the Cu(II)-materials still maintain their ability to convert S-nitrosothiols to NO. Similarly, polymer films made with the Cu(II)-complexes then stored under ambient conditions for >1 month are able to generate NO at the same level as the fresh films.

### Example 4

### Preparation of Cu(II) Complex/Polymer Composition

Tecoflex polyurethane (SG-80A) (TPU) is dissolved in appropriate solvents (e.g., tetrahydrofuran (THF)) and adding 5 mg of a Cu(II)-complex per 50 mg polymer and shaking overnight to obtain a clear, slightly yellow polymer solution. The polymer cocktails is cast into 2.5 cm diameter Teflon rings with a Teflon base to form 10 μm-thick membrane. The membranes are cured overnight, covered. Membranes without the Cu(II)-complexes are be prepared similarly. Nitric oxide generation from the Cu(II)-complex polymer and control membranes are measured for three days in pH 7.4 buffer solution with added S-nitrosothiols, at 37° C. by chemiluminescence, using a calibrated Sievers Nitric Oxide Analyzer (NOA), model 280i. New buffer solution is added to the reaction vessel each 12 hours during the experiment. The control membranes are used to detect background decomposition of S-nitrosothiols.

### Example 5

### Leaching

The theoretical octanol/water partition coefficient of the Cu(II)-ligand complex is estimated to be 10¹¹ based on calculations by the ChemDraw™ computer program. Although the complex is 10¹¹ times more likely to remain within the polymer phase, the potential does exist for the ligand and/or copper ions to leach from the matrix into the soaking solution (eventually blood); thereby limiting the lifetime of NO generation by polymers doped with this complex as well as causing potential toxicity concerns (although a small amount of copper is often a component of vitamin supplements). Thus, the degree of leaching as a function of time is measured by soaking polymer films containing the complex in PBS and other media more closely approximating whole blood (such as serum). This leaching is a function of the partition coefficient of the complex (k^{pot}), as well as its diffusion coefficient (D_{complex}) within the polymer matrix.

The formulations are evaluated for leaching of the Cu(II)-complex and copper after 3 days of soaking in PBS buffer containing S-nitrosothiols. Polymer films containing the Cu(II)-complexes are soaked in oxygenated PBS buffer for a period of time (i.e., 1, 2, and 3 days) at 37° C. The films are removed from the PBS solution and the soaking bath analyzed for the presence of copper via ICP-MS at detection limit of 1 μM.

### Example 6

### Coating and Mechanical Testing of the Outer Membrane

Mechanical properties of the sensor membrane is evaluated by tensile test using an Instron (Canton Mass.) mechanical tester to determine the elasticity (Young's Modulus) and the maximum tensile stress of the membrane doped with the Cu(II)-complexes. These membranes are compared to the polymer membranes that do not contain the Cu(II)-complexes. The integrity and efficacy of the coating are evaluated via peeling and scratch tests as well as SEM (Scanning Electron Microscopy). The 90° rigid substrate coating peel test is executed on Instron using peel test fixture by bonding an extremely high-strength peel tape material, and then peeling at an exact 90° angle. The angle is maintained by a slider that is pulled along synchronously with the pull-up rate, while continuously recording the progressive peel force. Scratch test is performed on the polymer formulations using a scratch tester to determine the durability of the coating. These mechanical properties are tested prior to soaking in buffer and with storage in PBS buffer (pH 7.4) at 37° C. for 3 days to look for any possible degradation of the coating over time.

### Example 7

### Partitioning of Glucose Through the Outer Coating

To ascertain whether glucose will be able to partition into the Cu(II) complex coating in order to react with GOx at the electrode surface, a diffusion cell arrangement is used, placing thin films of the outer polymer matrix imbedded with the Cu(II)-complex between two given volumes of stirred PBS solutions. A fixed high concentration of glucose is added to one compartment, and samples are taken from the recipient side at various times and analyzed for the presence of the glucose. The rate of appearance of glucose on the recipient solution side is proportional to Dₖ, the diffusion coefficient of glucose (D) and its partition coefficient (k) into the polymeric film. Detection of glucose in the receiving solution is accomplished by utilizing a glucose sensor in a three electrode configuration (prepared in house) with an applied potential of +0.7 V.

### Example 8

### Optimization of Glucose Oxide to the Electrode Surface

There are several methods for immobilization of enzymes, especially GOx. Immobilization methods include: entrapment of the enzyme, covalent bonding of receptors on surfaces activated by means of bifunctional groups or spaces and bulk modification of the electrode material. The enzyme can be directly immobilized onto the electrode surface from a solution of glucose oxidase (Aspergillus niger, 260 U/mg, GO3AC, Biozyme, San Diego, Calif.), and bovine serum albumin and cross-linked with glutaraldehyde (Sigma). The second method involves doping high quantities of GOx into a solution of Tecophilic PU then depositing an aliquot of the solution to the electrode surface and allowing the surface to dry.

### Example 9

Amperometric measurements are made with a CH instrument Model CHI900 electrochemical analyzer and used to evaluate the analytical performance of the glucose sensors. Control and NO-generating sensors are immersed in PBS for 1 h and polarized at +0.7 V (Vs. Ag/AgCl) to obtain a stable baseline. Calibration curves of the response of the sensor to varying glucose concentrations are obtained by injecting standard solutions of glucose into the PBS buffer with constant stirring and measuring the current output. To assess the sensitivity and stability of the sensor with time, calibrations are performed every 24 h for glucose concentrations up to 30 μM since this value exceeds physiological levels even for seriously ill patients. The response times and calibration after each 24 h period are compared to the initial specifications to determine sensor drift or a decrease in the response rate.

### Example 10

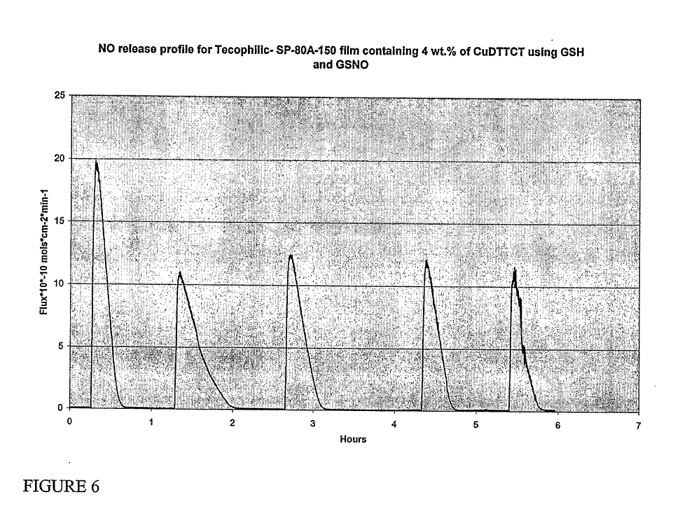

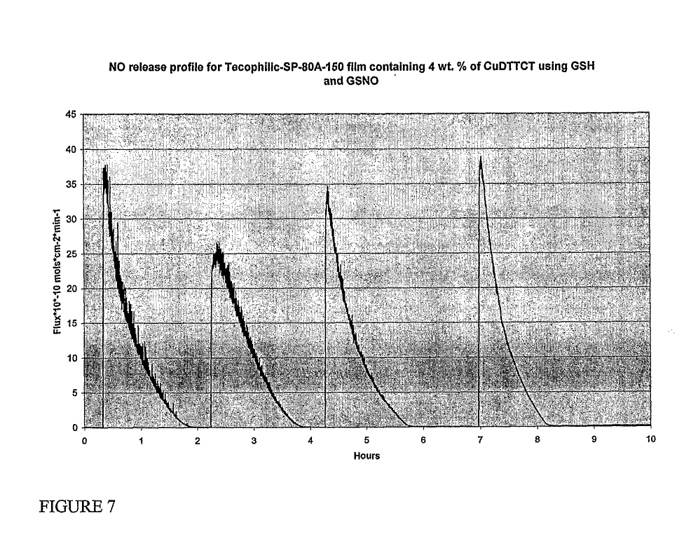

FIGS. 6 and 7 show the NO generating profile for a tecophilic polyurethane (198.1 mg of SP-80A-150) film containing 4 wt. percent of CuDTTCT. Approximately 78.9 uL of a 0.0057 M solution of L-glutathione and 27.8 uL of a 0.0054 M solution of nitroso-thiols is added to the copper film. FIG. 6 indicates a consistent generation of NO as the copper film is exposed to the nitroso-thiol solutions on day 2, and FIG. 7 indicates a consistent generation of NO as the copper film is exposed to the nitroso-thiol solutions on day 3.

### Example 11

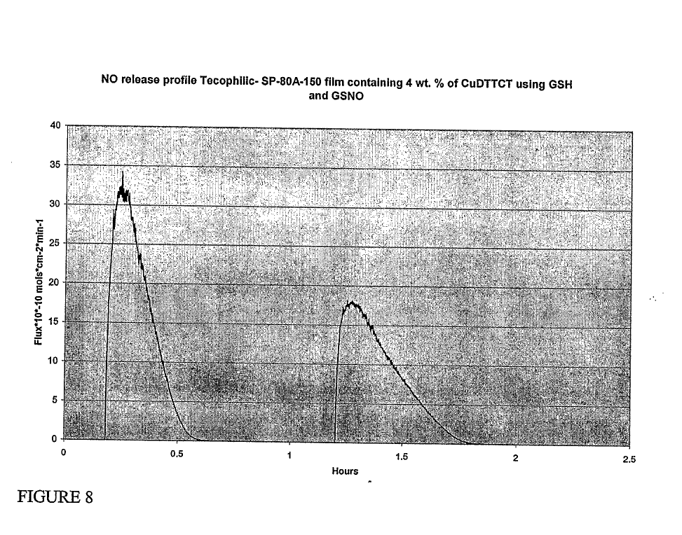

FIG. 8 shows the NOgenerating profile for a tecophilic polyurethane ( 198.1 mg of SP-80A-150) film containing 4 wt. percent of CuDTTCT after film was soaked in pig blood for 3 days. Approximately 78.9 uL of a 0.0057 M solution of L-glutathione and 27.8 uL of a 0.0054 M solution of nitroso-thiols was added to the copper film. FIG. 8 indicates the NO generation after film is soaked in blood and then exposed to the nitroso-thiol solutions.

### Example 13

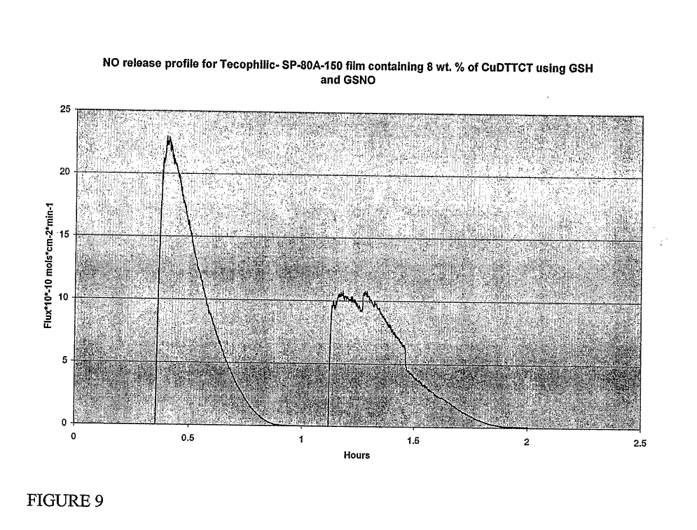

FIG. 9 shows the NO generating profile for a tecophilic polyurethane (206.1 mg of SP-80A-150) film containing 8 wt. percent of CuDTTCT after film was soaked in pig blood for 3 days. Approximately 78.9 uL of a 0.0057 M solution of L-glutathione and 27.8 uL of a 0.0054 M solution of nitroso-thiols was added to the copper film. FIG. 9 indicates NO generation after film is soaked in blood and then exposed to the nitroso-thiol solutions.

### Example 14

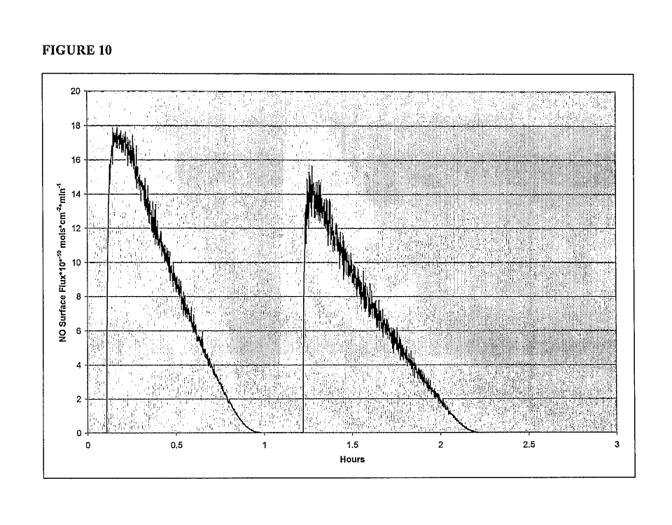

FIG. 10 shows the NO generation profile for a tecophilic polyurethane (206.3 mg SP-80A-150) film containing 4 wt. % of a NO-generating agent after the film was soaked in a 40 uM glutathione solution for over a month. For testing, approximately 78.9 uL of a 0.0057 M solution of L-glutathione and 27.8 uL of a 0.0054 M solution of nitroso-thiols was added to the NO generating film. FIG. 10 indicates that after exposing the film to a glutathione solution for over a month the film still maintains NO generation levels higher than that of physiological conditions.

### Example 15

### Sensor Fabrication and Performance

The control sensor configuration consists of a Pt working wire electrode and a Teflon™-coated Ag/AgCl reference electrode. The sensor sleeve is constructed from a Tecoflex EG80A tubing, with an inner diameter of 0.043″ and an outer diameter of 0.057″, cut to a length of 2.2 cm. Platinum and silver wires are inserted into the sleeve and affixed inside of it with epoxy. Care is taken that the electrodes did not touch one another. After the epoxy has dried, the end of the sensor is clipped with a razor blade and immersed in an aqueous 0.1 M FeC13/0.1 M HCl solution to form the Ag/AgCl reference electrode.

The enzyme layer is formed as follows: 0.5 μL 1% glutaldeyde is dripped onto the tip of the sensor with a micropipette, and allowed to dry. 3 μL of glucose oxidase solution (10 mg glucose oxidase, 8 μL 1% BSA, 200 μL H20)—is added to the tip and allowed to dry. This is followed by a second 0.5 uL 1% glutaldehyde and after drying, 3 uL glucose oxidase solution. A third coat of 3 uL glucose oxidase is added after the second has dried. The polymer layers are added by dipping the sensor in a 40 mg Tecophilic PU SG80A/mL THF solution, letting it dry for ten minutes, and then dipping the sensor again in the same solution and letting it dry for four to five hours.

Sensors are polarized to +0.7 V in 15 mL PBS using a CHI800B electrochemical analyzer for 1 hour. Volumes of 150 mM D-(+)-glucose in PBS are added to the sensor fluid to increase the glucose concentration by 2.5 mM for every volume added, for concentrations from 2.5 to 30 mM. These concentrations are chosen to broadly bracket the possible physiological concentrations in humans. (Ganong, William F. Lange's Review of Medical Physiology. 22^{nd} edition. McGraw-Hill, New York. 2005.)

Final formulations of the enzyme and polymer layers are decided upon based the magnitude and linearity of the sensor response to glucose over the range of concentrations given above. Once a functional 2.2-cm sensor is found, 30-cm control and NOGEN-coated sensors are constructed.

### EQUIVALENTS

The present invention provides among other things, compounds, compositions, polymers, and methods. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. To the extent that any U.S. Provisional Patent Applications to which this patent application claims priority incorporate by reference another U.S. Provisional Patent Application, such other U.S. Provisional Patent Application is not incorporated by reference herein unless this patent application expressly incorporates by reference, or claims priority to, such other U.S. Provisional Patent Application.

Also incorporated by reference are the following:

Patents and patent applications
US20020115559-A1

We claim:

1. An analyte sensor suitable for implantation in a patient comprising:
an electrode surface comprising an enzyme; and
a biocompatible analyte permeable composition comprising a nitric oxide generating agent, wherein said composition generates nitric oxide when in contact with nitrosothiols.

2. The analyte sensor of claim 1, wherein said enzyme is an oxidase.

3. The analyte sensor of claim 1, wherein said composition further comprises a pharmaceutically effective agent.

4. The analyte sensor of claim 2, wherein the enzyme is glucose oxidase.

5. The analtye sensor of claim 1, wherein said composition substantially prevents biofouling of said sensor for three days or more after implantation.

6. The analyte sensor of claim 1, wherein said sensor is capable of continuously measuring blood glucose levels in a patient for 3 days or more.

7. The analyte sensor of claim 1, wherein said sensor is subcutaneously implantable.

8. The analyte sensor of claim 1, wherein said composition is disposed on said electrode surface.

9. The analyte sensor of claim 1, wherein said analyte permeable composition can substantially exclude at least one of: ascorbate, urate, cysteine or paracetamol.

10. The analyte sensor of claim 1, wherein said analyte permeable composition is substantially glucose permeable.

11. The analyte sensor of claim 1, wherein said nitric oxide generating agent is a metal ion binding moiety.

12. The analyte sensor of claim 11, wherein said metal ion binding moiety is a Nₓ-donor ligand where x is 2,3,4,5,6,7,8or9.

13. The medical device of claim 11, wherein said nitric oxide generating compound is a S_{y}-donor ligand where y is 2, 3, 4, 5, 6, 7, 8 or 9.

14. The analyte sensor of claim 11, wherein said nitric oxide generating agent is a metal-ligand complex comprising said metal ion binding moiety and a metal ion that is redox active.

15. The analyte sensor of claim 14, wherein said metal ligand complex is a metal-cyclen complex or a metal-cyclam complex.

16. The analyte sensor of claim 15, wherein said metal-cyclen complex is a copper-cyclen complex or a copper-cyclam complex.

17. The analyte sensor of claim 14, wherein said metal-ligand complex is a N₄ donor type macrocycle.

18. The analyte sensor of claim 1, wherein the nitric oxide generating agent is represented by Formula I: 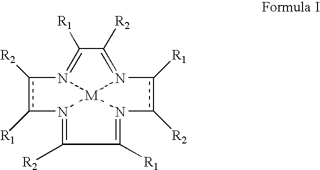
wherein:
R₁ and R₂ represent each independently: alkyl, alkenyl, alkynl, H, or any R₁ and R₂ together form an aryl or an heteroaryl;
------- represents a bond with any bond order;
M is a metal; and salts thereof.

19. The analyte sensor of claim 18, wherein M is metallic ion selected from the group consisting of Ca, Cu, Zn, Co, Mn, Al, Fe, V, Cr, and Ti.

20. The analyte sensor of claim 18, wherein the nitric oxide generating compound is a Cl, I, F, or Br salt of Formula I.

21. The analyte sensor of claim 18, wherein M is Cu(II).

22. The analyte sensor of claim 1, wherein said nitric oxide generating agent is selected from at least one of: Cu(II)-dibenzo[e,k]-2,3,8,9-tetraphenyl-1,4,7,10-tetraaza-cyclododeca-1,3,7,9-tetraene, Cu(II) dibenzo[e,k]-2,3,8,9-tetramethyl-1,4,7,10-tetraaza-cyclododeca-1,3,7,9-tetraene, or Cu(II) dibenzo[e,k]-2,3,8,9-tetraethyl-1,4,7,10-tetraaza-cyclododeca-1,3,7,9-tetraene.

23. A biocompatible membrane for use in an analyte sensor comprising a nitric oxide generating agent, wherein said analyte sensor is capable of continuously measuring blood glucose levels for 3 days or more.

24. The biocompatible membrane of claim 23, further comprising:
a first layer comprising immobilized glucose oxidase;
a second layer disposed on the first layer, wherein said second layer comprises a metal-ligand complex.

25. The biocompatible membrane of claim 24, wherein said membrane has a thickness of about 0.1 μpm to about 15 μm.

26. An analyte sensor comprising:
an electrode surface comprising an enzyme;
an analyte permeable composition; and
a biocompatible composition comprising a nitric oxide generating agent, wherein said composition generates nitric oxide when in contact with nitrosothiols

27. A method for detecting glucose levels in the blood of a patient over 2 days or more, wherein said method comprises:
instilling a glucose sensor that comprises a biocompatible analyte permeable composition subcutaneously into a patient; wherein said composition comprises a nitric oxide generating agent.

28. A glucose detection kit comprising
a glucose sensor comprising a nitric oxide generating agent; and
instructions for use.

29. The use of a nitric oxide generating agent for a blood glucose sensor.
